(19) United States
(12) Patent Application Publication
(10) Pub. No.: US 2007/0178122 A1
(43) Pub. Date: Aug. 2, 2007

(54) Fusion proteins comprising modified allergens of the ns-ltps family, use thereof and pharmaceutical compositions comprising the same

(76) Inventor: Domenico Geraci, Palermo (IT)

Correspondence Address: NIXON & VANDERHYE, PC, 901 NORTH GLEBE ROAD, 11TH FLOOR, ARLINGTON, VA, 22203, US

(21) Appl. No.: 10/590,940

(22) PCT Filed: Feb. 28, 2005
(86) PCT No.: PCT/IB05/50714

§ 371 Date: Sep. 26, 2006

(30) Foreign Application Priority Data
Feb. 27, 2004 (IT) RM2004A000103

(51) Int. Cl. A61K 39/35; C07H 21/04; C12P 21/06; C07K 14/415

(52) U.S. Cl. 424/275.1; 435/69.1; 435/419; 435/468; 530/370; 536/23.6

(57) ABSTRACT

The present invention relates to fusion proteins comprising different allergens of the ns-LTPs family, and their use in the prevention and the treatment of allergic symptoms associated to said allergens. In particular, fusion proteins in the form of heterodimers comprising the major allergens of Parietaria judaica, Parj 1 and Parj 2, are described. Methods of preparation of the fusion proteins by expression in genetically modified host cells and pharmaceutical compositions comprising said fusion proteins are described as well.

### FIELD OF THE INVENTION

The present invention lies within the fields of the prevention and the treatment of allergic symptoms associated to allergens belonging to the non-specific Lipid Transfer Protein (ns-LTPs) family.

### STATE OF THE ART

ns-LTPs proteins are small proteic molecules of approximately 10 KDa that demonstrate high stability, and are naturally present in all vegetal organisms studied to date. These proteins are characterised by their ability to transport lipids through membranes in vitro.

In several species they have also been identified as allergens, as in the case of the Rosaceae Prunoideae (peach, apricot, plum) and Pomoideae (apple), as in the Urticacee like Parietaria. The genus Parietaria includes 5 species, P. Judaica being the most allergenic one.

The allergic reaction, also called Type I hypersensitivity, is induced by an IgE-mediated response to environmental antigens, usually innocuous and present, e.g., in pollen grains. The IgE/Allergens interaction is the initial event of a cascade of reactions leading to the release of mediators, like histamine, responsible for the allergic symptomatology. When localised at skin level, the release of histamine causes itch, erythema and edema, whereas when generalised it causes bronchoconstriction at the brochopulmonary level and impressive phenomena involving the cardiovascular system.

The most common allergic illnesses are rhinitis, conjunctivitis, urticaria, angioedema, eczema, dermatitides, asthma and, in extreme cases, anaphylactic shock.

Recombinant DNA technology allowed the isolation of various allergens of the ns-LTPs family, among them of the major allergens of Parietaria denominated Parj1 and Parj2 (Colombo, P., et al., The allergens of Parietaria Int Arch Allergy Immunol. 2003 March;130(3):173-9, Review).

Parj1 is a protein of 176 amino acids and a molecular weight of 18.450 Da. The N-terminal sequence exhibits an amino acid composition characteristic of signal sequences of glycosilated proteins. The mature protein is composed of 139 amino acids, with a molecular weight of 14726 Da. It is a major allergen, since it binds more than 90% of sera of subjects allergic to Parietaria judaica. (Costa et al. cDNA cloning, expression and primary structure of Par jI, a major allergen of Parietaria judaica pollen. FEBS Lett., Mar. 21, 1994; 341 (2-3):182-6.)

Parj2 is a protein of 133 amino acids, and it contains a signal peptide of 31 amino acids. The mature protein is composed of 102 amino acids, with a molecular weight of 11344 Da. It exhibits a 45% homology with the Parj1 at the amino acid level and is it also a major allergen, since it reacts with the near-totality of the sera of allergic subjects (Duro, G., et al., cDNA cloning, sequence analysis and allergological characterization of Parj 2.0101, a new major allergen of the Parietaria Judaica pollen. FEBS Lett, 1996. 399(3): p. 295-8).

In spite of the their structural homology, Parj1 and Parj2 are anyhow two independent allergens containing epitopes them also independent, as highlighted by cross-inhibition experiments. When a pool of sera of allergic subjects is preincubated with the recombinant Parj1 and Parj2 allergens, IgE binding to the 10-14 kDa region is totally inhibited, suggesting that only these two allergens are present in this region and that together they are capable of inhibiting the majority of specific IgE against Parietaria judaica allergens (Table FIG. 8).

The Parj1 and Parj2 allergens exhibit all the characteristics of the ns-LTP and the structural model of the Parj1 was determined using the crystal of the ns-LTP from soy seed as reference. According to said model, both molecules have 4 disulphide bridges, in the order: 4-52, 14-29, 30-75, 50-91. All ns-LTP proteins considered, when suitably aligned as illustrated in FIG. 2 of Pat. App. WO-A-02/20790, contain four disulphide bridges between cysteine residues in positions corresponding to the aboveindicated positions of Parj1 or Parj2.

By applying a strategy of site-specific mutagenesis, it has previously been demonstrated the relevance of the disulphide bridges in the formation of the IgE epitopes (WO-A-02/20790) and the existence of a dominant epitope in the loop1 region comprised between amino acids 1 and 30. (Colombo, P., et al., Identification of an immunodominant IgE epitope of the Parietaria Judaica major allergen. J. Immunol, 1998. 160(6): p. 2780-5).

From a therapeutic standpoint, various pharmacological treatments of the allergic symptomatology do exist, whereas the sole preventive therapy is represented by the specific immunotherapy (SIT). This therapy foresees the subcutaneous administration of diluted quantities of allergen to the patient so as to suppress the specific reaction towards the allergen. However, the majority of the commercial protein extracts used therefor are anyhow crude extracts, mixtures of several components in which a precise standardization of the allergenic component is difficult. Thus, the SIT strategy can entail the administration of allergenic components towards which the patient is not sensitive, inducing the production of IgEs specific towards other components of the extract. Moreover, the administration of the total allergen entails the risk of side effects, which could even cause anaphylactic shock. In order to eliminate some of the disadvantages described hereto, there have been developed alternative molecules with reduced side effects, i.e., capable of not interacting with the IgEs while maintaining the capability of immunosuppressing the T response and capable of stimulating the production of IgG immunoglobulins.

The preceding work of the present Authors, disclosed in Pat. App. WO-A-02/20790, lies within the same purview. By genetic manipulation, there were produced variant forms, or muteins, of ns-LTP allergens, characterised by the partial or total elimination of the disulphide bridges typical of the native protein. These muteins have a reduced allergenic activity with characteristics such as to make them useful in SIT as molecules substituting the native proteins.

A different approach is that described by B. Linhart in Pat. App. EP-A-1 219 301. In this case, there are described hybrid polypeptides containing at least two wild type allergenic proteins or fragments thereof. Working on Phlenum pratense pollen allergens Phl-p or on parasite allergens Der, Linhart observes a reduced allergenicity of the hybrid peptides.

However, there remains a need for novel tools suitable for the treatment of specific allergic forms, like those caused by the ns-LTPs proteins, tools combining the characteristics of reduced allergenicity with high immunotherapeutic effectiveness and easy accessibility with regard to both their preparation and their use. Scope of the present invention is to satisfy this need.

### SUMMARY OF THE INVENTION

Epidemiological studies demonstrated that subjects generally allergic to various plants belonging to the same genus, or to the same species, or even to a single specific plant variety, in the majority of cases have IgEs against plural allergens produced by the plant. E.g., in the case of subjects allergic to Parietaria judaica, those usually have IgEs against both of the major allergens, i.e., the proteins Parj1 and Parj2. As highlighted in FIG. 8 (Table) in a binding inhibition assay between the human IgEs in sera of PJ allergic patients and an extract of P. judaica, the percentage of inhibition induced by the mixture of wild type Parj1 and Parj2 is usually higher than that induced by the individual allergens. Hence, any one therapeutic formulation suitable for the treatment of allergy should comprise all the main allergens, or muteins thereof, produced by one or more plants responsible for the allergy.

The present invention is based on the unexpected discovery that hybrid proteins, obtained by the fusion of the polypeptide sequences of plural allergens in mutated form, have characteristics that are advantageous both from the therapeutic and preparative standpoint, as well as with regard to the management of the medicament compared to mere mixtures of plural allergens.

Main object of the present invention are fusion proteins comprising amino acid sequences of different allergens belonging to the ns-LTPs protein family, wherein said sequences lack one or more of the four disulphide bridges present in the sequence of the wild type allergens, in particular lacking at least one disulphide bridge in the amino-terminal region comprised between the amino acid residues 1 and 30. The amino acid sequence of each of the allergens is independently mutated by elimination or substitution of one or more cysteine residues involved in the formation of a disulphide bridge, though maintaining essentially the same length of the sequences of wild type allergens.

The allergens of the invention are produced by plants belonging to the same genus, or to the same species, or preferably to the same vegetal variety. In a preferred embodiment the fusion protein is a heterodimer protein comprising the amino acid sequences of two different allergens, e.g., allergens of the same plant like the allergens Parj1 and Parj2 of the Parietaria Judaica species.

In this case, the amino acid sequences of Parj1 and Parj2 allergens will both be independently modified by substitution of cysteine residues with residues not capable of forming a disulphide bridge in positions 29 and 30 or 4, 29 and 30 or 29, 30, 50, 52.

Further objects of the invention are a nucleotide sequence comprising the DNA coding for the fusion protein of the invention, an expression or cloning system comprising the nucleotide sequence at issue flanked by suitable sequences for controlling, promoting and regulating the expression, and a host cell transformed by means of said expression or cloning system.

Other objects of the invention are medical uses of the fusion protein, in particular as hypoallergenic immunological agent in the specific immunotherapy (SIT) treatment of allergies, as well as pharmaceutical compositions comprising the fusion protein and a pharmaceutically acceptable excipient.

Further objects of the invention are methods of preparation of the fusion protein in which suitably mutated amino acid sequences of different allergens are produced and bound directly or via a linker for chemical synthesis or by expression, in the form of fusion protein, in genetically modified host cells, as well as methods of preparation of the pharmaceutical compositions.

The fusion molecules according to the invention provide indisputable advantages. First of all, they exhibit a capability of interacting with the IgEs that is markedly reduced with respect to: the individual allergens or mixtures of wild type allergens, as it is apparent from a comparison of the data reported in the Table of FIG. 5 (PjEDcys lane) to those in the Table of FIG. 8; the individual modified allergens, as it is apparent from a comparison of the Table of FIG. 5 with the Table reported in FIG. 9; or to the heterodimer of wild type (Wt) allergens, as again apparent from the Table of FIG. 5. Moreover, cytofluorometric analysis of peripheral blood cells CD3+ proliferation demonstrates that this reduced allergenicity is advantageously accompanied by an unaltered or even enhanced immunogenic capability, as illustrated in FIG. 7, panels B and C, and as resumed by the Table in the same FIG. 7. Not only the reduction of the allergenic characteristics of the heterodimer, accompanied by a marked immunogenic capability with respect to the individual wild type or mutated allergens or to mixtures thereof, was unforeseeable; already the mere maintenance by the hybrid protein of the typical features of the individual modified allergen was a characteristic in no way expectable.

Moreover, with respect to the mere mixture of allergens, or of muteins thereof, the heterodimers of the invention entail the further advantage of being producible via a single process, of course simplifying all the procedures of production, control, storage, authorization to sale and use, with an indisputable saving of times and material and financial means.

### BRIEF DESCRIPTION OF THE FIGURES

FIG. 1: Nucleotide sequence of the Parj2-Parj1 dimer in its form mutated on the residues in positions 10, 85, 88, 122, 397 and 400. In all the positions indicated the nucleotide T was substituted with the nucleotide A (in bold type). The “GGATTC” spacer between the sequences coding for the two allergens Parj2 and Parj1 is also highlighted in bold type (SEQ ID NO:3)

FIG. 2: Amino acid sequence of the Parj2-Parj1 heterodimer in its form mutated on the residues in positions 4, 29 and 30 in each of the two molecules (PjEDcys). The amino acid sequence is expressed in one-letter code. Underlined amino acids indicate the substitutions effected. Amino acids in bold type and italics indicate the amino acids glycine and phenylalanine introduced with the cloning. (SEQ ID NO 4)

FIG. 3: ELISA detection of the binding capability of the Parj2 allergen as compared to the binding activity of the Parj2/4,29,30 mutant. Lines with black squares indicate the sera of Pj allergic subjects, the line with white squares indicates a sera of a Pj non allergic patient.

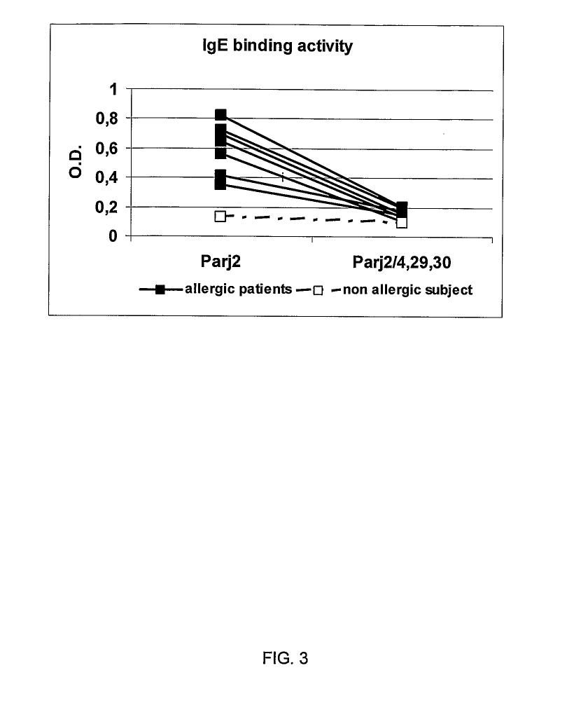

FIG. 4: Panel A: schematic representation of the PjEDcys mutant. Panel B: Western blot Analysis carried out by using the recombinant Parj1 proteins (lane A), Parj2 (lane B), Parj2-Parj1 diner (Wt dimer clone) (lane C), PjEDcys (lane D) with a serum of Pj allergic subject. Panel C: the same membrane of Panel B incubated with a probe specific for the histidine tail.

FIG. 5: ELISA detection of human IgEs binding inhibition by the Wt dimer and PjEDcys recombinant proteins, using a Parietaria judaica crude extract as antigen and 5 sera from Pj allergic patients.

FIG. 6: Testing of histamine release from blood of Pj allergic patients. The antigens used were: an equimolar mixture of the two rParj1 and rPar2 allergens (line with rhombs, denominated rPj1+rPj2) and the PjEDcys mutant (line with squares). On the x-axis the amounts of protein used, and on the y-axis the percentage of histamine released with respect to the percentage of total histamine of the patient's mastocytes are reported.

FIG. 7: Cytofluorometric analysis of the proliferation of CD3+ cells from PBMC.

CSFE-labeled cells were stimulated with a solution containing a mixture of the Parj1 and Parj2 allergens (panel B) and a mixture containing an equimolar amount of the PjEDcys hybrid (panel C). Panel A shows the unstimulated control culture.

The bottom table resumes the percentage of CD3+ cells capable of proliferating after antigenic stimulation in 5 allergic subjects. Numbers indicate the percentages of stimulation subtracted of the unstimulated cells.

FIG. 8: The table reports the results of an ELISA test of human IgEs binding inhibition by individual Wt allergens or mixture thereof.

FIG. 9: The table reports the results of an ELISA test of human IgEs binding inhibition by the mutated allergens PjA, PjB, PjC and PjD described in WO-A-02/020790.

### DETAILED DESCRIPTION OF THE INVENTION

The peptide sequences of wild type ns-LTPs allergens produced by various plants, like Parietaria judaica (Parj1 and Parj2), soy, lyces, ricco, tobacco, orysa, mais, spiol and wheat are reported in Pat. App. WO-A-02/20790. All molecules have four disulphide bridges between eight cysteine residues in highly conserved positions corresponding, when suitably aligned, to positions 4, 14, 29, 30 50, 52, 75 and 91, of the Parj1 and Parj2 molecules. The cysteine residues involved in disulphide bridges are the couplings 4-51, 14-29, 30-75 and 50-91.

Muteins of these allergens with a reduced capability of forming disulphide bridges are molecules in which one or more cysteine residues involved in the —SS— bond have been eliminated or substituted with other residues not capable of taking part in the binding, yet without sterically altering the spatial conformation of the molecule, e.g., Asn, Ser, Thr, Ile, Met, Gly, Ala, Val, Gln or Leu. Preferred muteins are obtained by elimination of two, three or four disulphide bridges; e.g., those corresponding to bonds 14-29 and/or 30-75 and/or 4-51 and/or 50-91 of the Parj1 or Parj2 molecule. Molecules mutated by substitution of the cysteine residue with a serine or alanine residue in positions 29, 30, or 4, 29, 30, or 29, 30, 50, 52 of Parj1 or Parj2, or in the corresponding positions of the other ns-LTPs allergens, exhibit the best properties of low allergenicity. Apart from the above ones, the muteins used in the invention exhibit no other modifications, and therefore maintain substantially unaltered the sequence, the length and the molecular weight of the wild type allergen.

The polynucleotide sequences coding for wild type allergens of the ns-LTPs family are available to the public on databanks like EMBL, or described in the state of the art. In particular, the nucleotide sequences of Parietaria judaica allergens are described in the abovementioned WO-A-02/20790 and in Duro, G., et al FEBS Letter 1996 (supra).

The preparation of the muteins of the invention is carried out via any known method suitable for the introduction of variations on individual amino acid residues along the polypeptide sequence of proteins. Usually, the variation is conducted via a site-specific point mutation at the level of the coding nucleotide sequence by DNA PCR method and using suitable synthetic oligonucleotides. The procedures are described, e.g., in the preceding application WO-A-02/20790.

The fusion proteins of the invention contain the amino acid sequences, suitably mutated, of allergens obtained from the same vegetal family, e.g., from fagaceae, urticaceae, oleaceae, composites or graminae; or from the same genus, e.g., Parietaria; or from the same species, e.g., P. judaica, officinalis or lusitanica; or, even better, from the same plant variety. Preferred fusion proteins are those comprising muteins of the main allergens of the Parietaria judaica, i.e., Parj1 and Parj2 or isoforms thereof known and deposited, e.g., in EMBL. The two proteins bound in an heterodimer can be modified according to the same scheme or to different schemes. Thus, the two proteins could contain disulphide bridges differing the one from the other in number and/or position. Preferred embodiments of the invention provide allergens individually and independently mutated in one or more of the positions corresponding to positions 4, 29 and 30 of the amino acid sequence of the major allergens of P. judaica. The fusion protein of the invention contains, e.g., the Parj1 and Parj2 allergens modified both in positions 29 and 30 or 4, 29 and 30 or 29, 30, 50, 52.

The preparation of the hybrid molecule- occurs by fusion of the two protein portions corresponding to the two allergens. Forming by synthesis a direct chemical bond e.g., peptidic, between the N-terminal and C-terminal residues of the two portions is viable; yet, the method used preferably implies the construction of a polynucleotide molecule coding for the allergenic proteins in fused form and suitably mutated in the desired positions (SEQ ID NO:1).

The two portions constituting the resulting heterodimer can be directly bound or separated by one or more amino acid residues. According to a scheme well-known to a person skilled in the art and detailed in the examples, clones containing the material coding for the suitably mutated individual allergenic proteins are amplified, digested with restriction enzymes and the coding fragments are bound and integrated into expression vectors. In order to facilitate its purification, the hybrid protein can optionally be expressed as fusion protein with a binding molecule exhibiting a specific affinity for a determined partner molecule; e.g., with a histidine tail in the amino terminal region allowing the purification on a His-trap column.

The cloning and expression systems used for the preparation of the fusion protein can be vectors suitable for prokaryotic or eukaryotic cells, e.g. the commercial pQE30 prokaryotic vector.

Pharmaceutical compositions suitable in the administration of the molecules of the invention are compositions in the form of aqueous, hydroalcoholic or oily solutions, of emulsions or suspensions, in aqueous or oily medium, or of liposome suspensions. Advantageously, the composition is formulated to attain a delayed release. Therefor, oily media or media containing suitable thickeners may be used. Besides the described formulations in liquid form, the compositions of the invention can be in semi-solid form like creams, pomades, gels or other forms suitable for topical application. Implants for subcutaneous application aimed at a prolonged release may be used as well. In this case the molecules of the invention are incorporated into a biodegradable or biodispersible polymer matrix under the action of the patient's natural enzyme system. Therefor, polymers such as polylactate or polyglycolate or polylactate/glycolate copolymers will be used.

The compositions at issue are formulated for a parenteral administration, in subcutaneous, intramuscular or intravenous use, for a topical administration on the skin or mucosae or for oral administration.

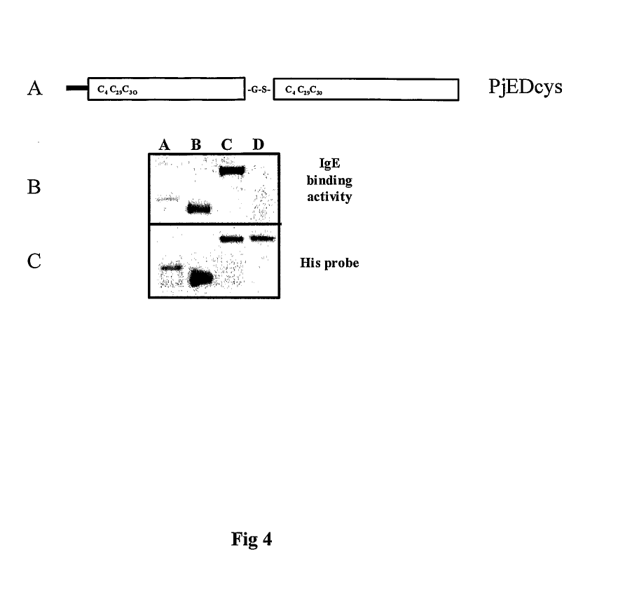

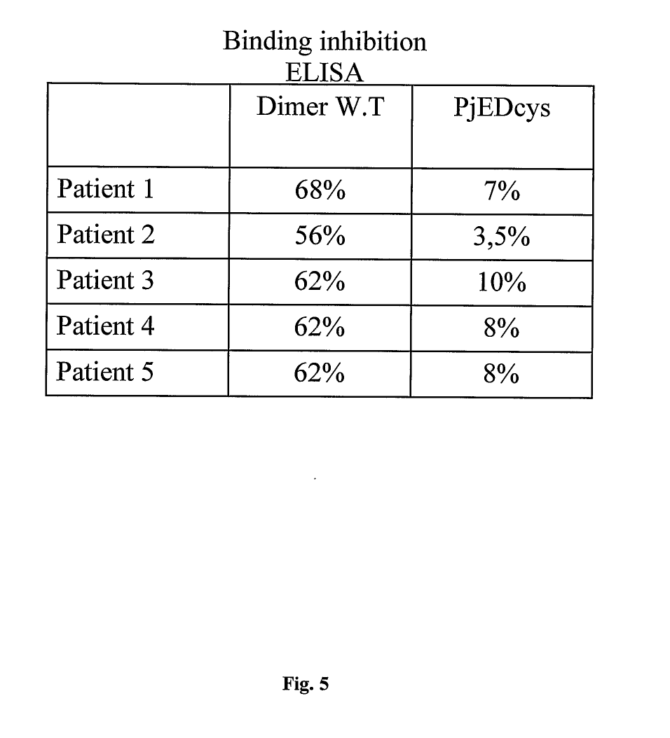

The fusion molecules of the invention are characterised by a marked hypoallergenicity with respect to the individual monomer allergens (FIG. 4, panel B and C) or with respect to a heterodimer molecule consisting of the Wt allergens (FIG. 5).

The hypoallergenic molecules of the invention find valid use as medicaments in the preventive and curative treatment of allergies caused by plural plant allergens, and in particular as desensitizers or immunosuppressants with reduced anaphylactic activity in SIT treatments. Exemplary allergic diseases that may be treated with the fusion protein of the invention are rhinitis, conjunctivitis, urticaria, angioedema, eczema, dermatitides, asthma, anaphylactic shock.

Lastly, the hypoallergenic fusion proteins of the invention find use for the preparation of DNA vaccines.

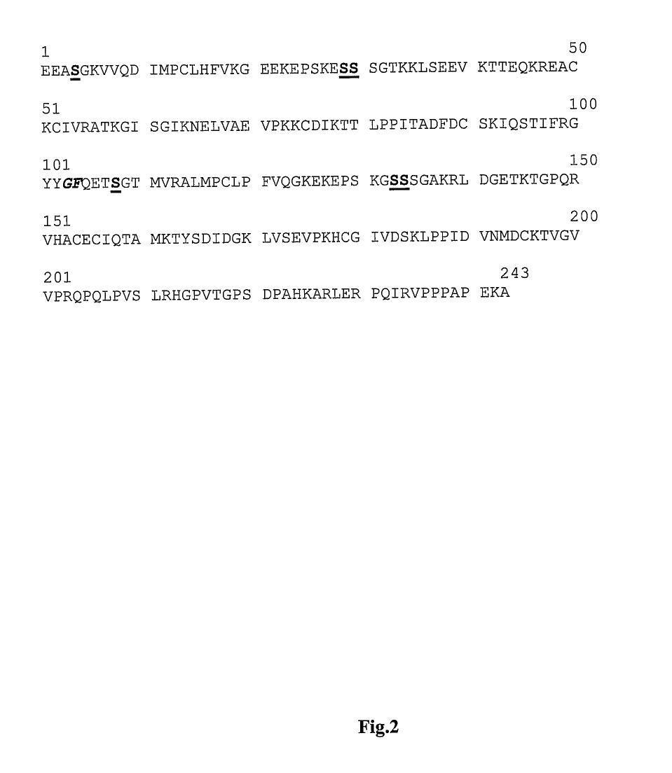

Hereinafter, there will be described the immunological characteristics of a specific heterodimer molecule formed by the fusion of the Par1 and Parj2 sequences and mutated in the respective positions 4, 29 and 30 (PjEDcys). In particular, such a protein was generated via the genetic fusion of the two polypeptides in the order Parj2/4,29,30-Par1/4,29,30. The fusion event caused the insertion of two additional amino acids (G and F) that do not interfere with the correct reading phase (FIGS. 2 and 4, panel A). Such a fusion protein was produced and purified by using a commercial prokaryotic expression system (fusion protein expression system pQE30, QIAGEN). FIG. 4, panel B, shows a Western-blot analysis from which it is inferred that the mutated dimer protein exhibits a reduced allergenicity (lane D) if compared to the IgE binding activity of the individual monomers (lanes A and B) or to a dimer molecule consisting of the wild type Parj1 and Parj2 allergens (lane C). This demonstrates that both the fusion event and the introduced mutation contribute to the attainment of the described hypoallergenicity. This data cannot be ascribed to a different amount of protein loaded on the gel, as it is demonstrated by panel C, where a probe specific for histidine tails was used. The reduced binding capability was then demonstrated by a technique independent from the preceding one where the heterodimer molecule PjEDcys was assayed for its capability of inhibiting human IgE binding to a Parietaria crude extract. In fact, FIG. 5 shows how this molecule inhibits IgE binding of 5 allergic patients with a value ranging from 3.5 to 10% and anyhow with values extremely reduced with respect to a dimer construct containing the Parj1 and Parj2 allergens in their native form (Wt dimer clone).

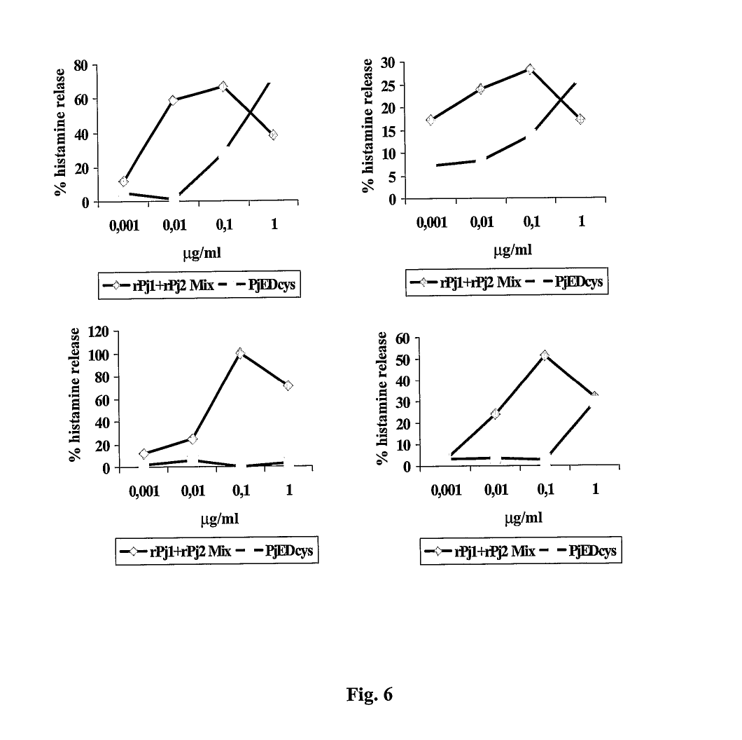

The effect of reduced binding capability was then demonstrated in a histamine release assay carried out on peripheral blood of allergic patients. The data reported in FIG. 6 show the histamine release percentages of the PjEDcys heterodimer as related to the release percentages of an equimolar mixture of the wild type Parj1 and Parj2 monomers. In all patients studied (N=4) the mutated molecule exhibits a marked reduction of the anaphylactic activity.

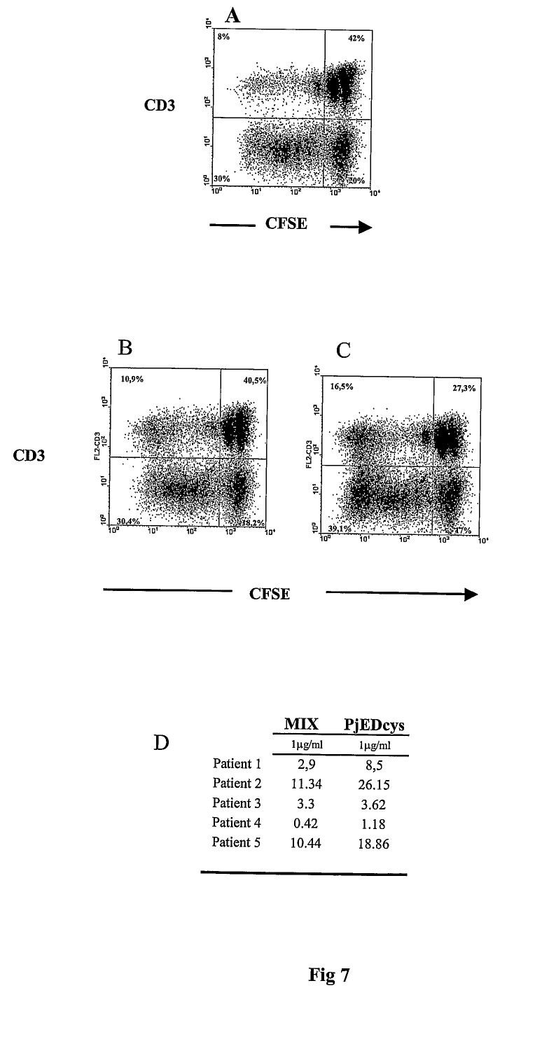

These variations at a structural level not only do not reduce the immunogenic capability of the molecule; on the contrary, they enhance its characteristics. In fact, in FIG. 7 it is reported the cell proliferation graph obtained by incubating PBMC purified from the blood of an allergic subject and after stimulation with the PjEDcys clone and with the mixture of wild type Parj1 and Parj 2 allergens. The mutations with regard to the cysteine residues and the fusion of the two proteins have no qualitative effect on the processing and the recognition of the allergen by blood T cells, yet they do markedly enhance the intensity thereof, as demonstrated by the Table (panel D) of the same FIG. 7.

Hereinafter, the invention will be illustrated by means of specific examples concerning the experimental steps of the preparation and the assessment of the immunological properties of the Parj2/4,29,30-Par1/4,29,30 fusion molecule. These examples have a merely illustrative purpose, in no way being limitative of the invention.

### EXAMPLE 1

### Construction of a Molecule Containing Genetic Information for the Parj2 Mutated in Cys 4, 29 and 30 (Parj2/4,29,30 Clone)

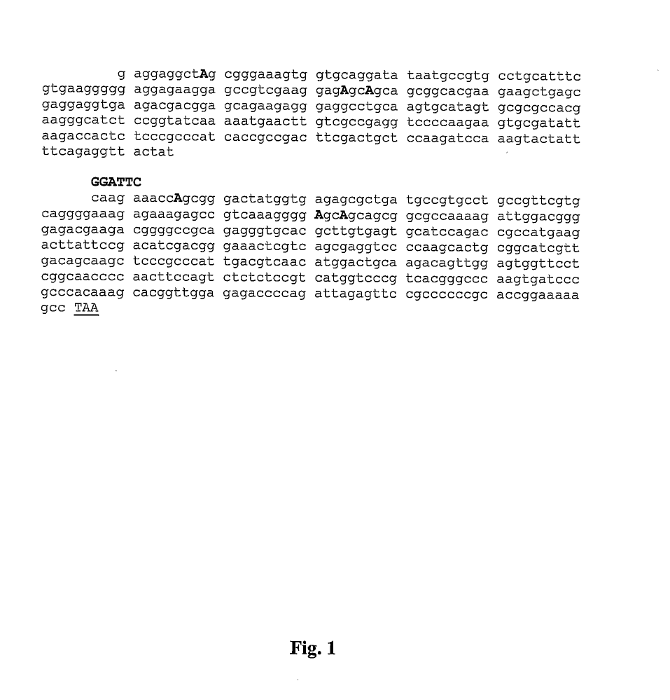

Site-specific mutagenesis with regard to cysteine residues in positions 29 and 30 was carried out using Transformer Site-Directed Mutagenesis kit (Clontech) following the manufacturer's instructions and using the synthetic oligonucleotide Pj2/29-30 5′ GAG AGC AGC AGC GGC AGC 3′ (SEQ ID NO 5). The clone, capable of expressing the wild type Parj2, was used as template for the mutagenesis and the cysteine residues in positions 29 and 30 were transformed into serine (Parj2/29-30 clone). Process success was confirmed by recombinant clone sequencing using the Sanger method. Mutagenesis of the cysteine residue in position 4 into serine was obtained by DNA polymerase chain reaction (PCR) using the synthetic oligonucleotides Pj2/4 5′ GTG GGA TCC GAG GAG GCT AGC GGG AAA GTG 3′ (SEQ ID NO 6) and Pj2 reverse 5′ GGG GGA TCC ATA GTA ACC TCT GAA 3′ (SEQ ID NO 7) and using the Parj2/29-30 clone as template. The DNA fragment thus obtained was cloned in the pQE30 expression vector (Qiagen). Analysis of the nucleotide sequence of the recombinant clone demonstrated substitution had occurred (see FIG. 1).

### EXAMPLE 2

### Construction of a Dimer Molecule Containing Genetic Information for the Parj1 and Parj2 Mutated in Cys 4, 29 and 30

The dimer molecule consisting of the Par1 and Parj2 allergens mutated in positions Cys4, Cys29 and Cys30, respectively, was obtained by a series of DNA amplification processes.

The Parj1 clone mutated in the cysteines at positions Cys4, Cys29 and Cys30 disclosed in patent n. WO 02/20790 (clone 29-30) was digested with BamH1 restriction enzyme.

The fragment containing the genetic information for the Parj2 mutated in positions Cys4, 29 and 30 (Parj2/4,29,30 clone) was subjected to DNA amplification process using oligonucleotides Pj2/4 and Pj2 reverse. The fragment thus generated was purified by agarose gel, digested with BamH1 restriction enzyme and incubated with a mixture containing the enzyme DNA ligase and the Parj 1 (29-30) clone previously linearised. Recombinant clones were purified and their nucleotide sequence determined by Sanger method. The hybrid protein thus constructed was expressed as fusion protein with one histidine tail in its amino terminal region to allow the purification thereof (PjEDcys clone) on affinity column. (See FIG. 2 and FIG. 4, panel A).

### EXAMPLE 3

### Induction and Purification of Recombinant Proteins

10 ml O/N culture were used for an inoculation in 400 ml of 2YT culture medium containing ampicillin and kanamycin to a final concentration of 100 μg/ml and 10 μg/ml, respectively. The growth occurs at 37° C. and under stirring. At +2 hour, IPTG to the final concentration of 1 mM was added to the culture and the growth proceeded for other 4 hours at 37° C. under stirring. Then, the bacterial culture was centrifuged at 5000 rpm for 15 min at 4° C. Pellet was resuspended in 5 ml/g Start buffer (10 mM Na phosphate pH7.4 and 6 M UREA) and the cells destroyed by using a sonicator. Then, recombinant proteins were definitively purified by using a His Trap column (Amersham) following the manufacturer's instructions. Eluted fractions were analysed on 16% polyacrylamide gel and fractions containing the recombinant protein were quantitatively assessed with Bradford method at the spectrophotometer after staining. Finally, proteins were desalted by using a Sephadex G-25 column (Pharmacia).

### EXAMPLE 4

### ELISA Assay for Assessing the Percentage of Inhibition of IgE Binding

ELISA test detection was carried out as described in Bonura et al. “Hypoallergenic variants of the Parietaria judaica major allergen Par j 1: a member of the non-specific lipid transfer protein plant family” Int Arch Allergy Immunol. 2001 September; 126 (1):32-40.), or as described in the abovementioned App. WO-A-02/020790. The concentration of the antigen used in each well is of 5 μg/ml. The patients tested (n=5) had a clear history of allergy to Parietaria judaica, and all tested positive to skin test using commercial products.

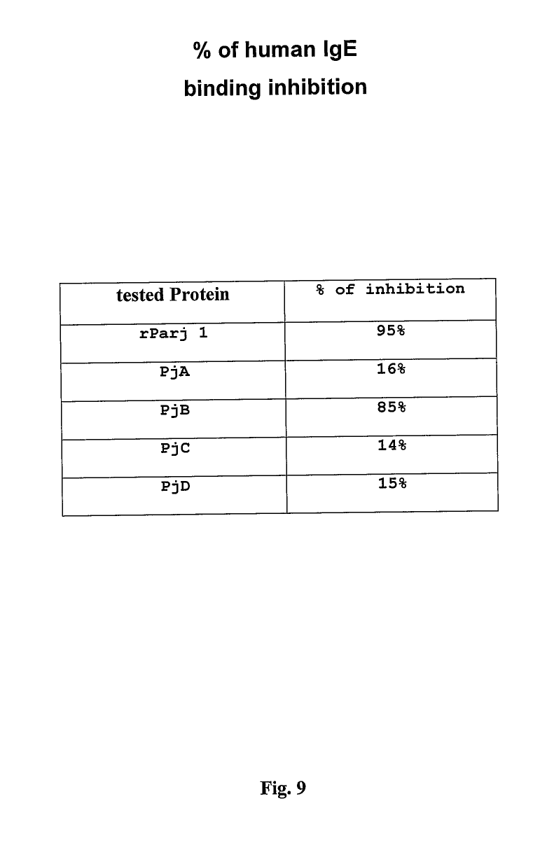

The results of the binding inhibition test with respect to the native protein and of the forms modified by elimination of two, three or four disulphide bridges are reported in the Table of FIG. 9.

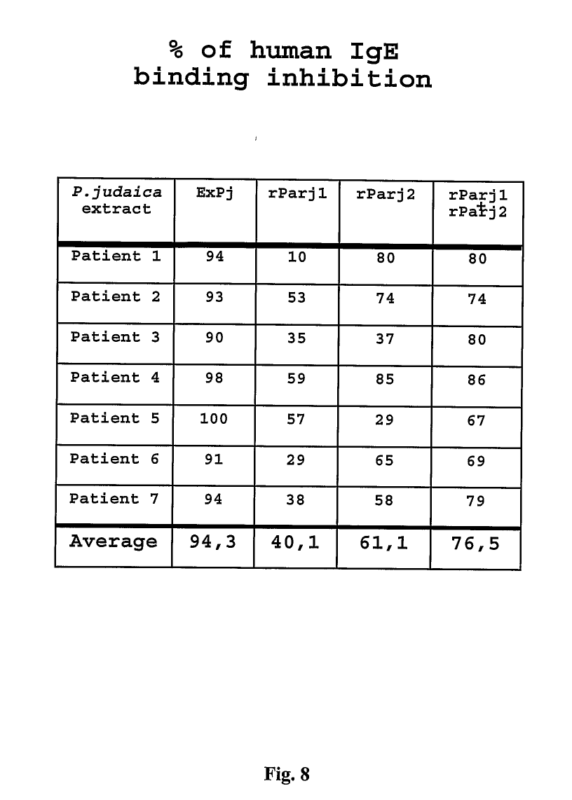

The results observed with the Wt heterodimer and the PjEDcys heterodimer are reported in the Table of FIG. 5. Lastly, the results observed with the Parietaria crude extract, the individual Wt allergens and the mixture thereof are reported in the Table of FIG. 8.

### EXAMPLE 5

### Histamine Release Assay

Histamine release assay was carried out using heparinised blood from Pj allergic patients and an allergen concentration scale ranging from 0.0001 and 1 μg/ml. Release protocol was carried out as previously described (Colombo, P., et al., Identification of an immunodominant IgE epitope of the Parietaria Judaica major allergen. J. Immunol, 1998. 160(6): p. 2780-5).

### EXAMPLE 6

### Study of PjEDcys-Induced CD3+ Cell Proliferation

PBMC from Pj allergic patients were purified and resuspended in 1×PBS pH 7.2 (1×10⁷ /ml) and labelled with Carboxy-Fluorescein Diacetate Succinimidyl Ester (CFDA-SE) to a final concentration of 5 mM for 5 min, at room temperature and in the dark. Cells were washed in complete RPMI (10% AB serum) and stimulated 7 days with a mixture containing 1 μg/ml Parj1 and Parj2 allergens and with an equimolar mixture of the PjEDcys hybrid. Then, the PBMC were stained with anti-CD3-PE antibody and analysed under cytofluorometry. The resulting data were analysed using the WinMDI 2.8 software. PBMC proliferation was determined by CFDA-SE (Carboxy-Fluorescein Diacetate Succinimidyl Ester) staining. Cytofluorometric analysis demonstrated that in the 5 subjects studied the PjEDcys hybrid is capable of stimulating a percentage of CD3+ cells far greater with respect to those stimulated by the mixture containing the allergens in monomer form (FIG. 7).

1-22. (canceled)

23. A fusion protein characterized in that it comprises the amino acid sequences of different allergens belonging to the non-specific Lipid Transfer Protein (ns-LTPs) family, in that said sequences lack one or more of the four disulphide bridges present in the sequences of the wild type allergens, at least one in the amino terminal region comprised between amino acid residues 1 and 30 and in that said sequences maintain essentially the same length as the sequences of wild type allergens.

24. The fusion protein according to claim 23, characterized in that the amino acid sequence of each of the allergens is independently mutated by elimination or substitution of one or more cysteine residues involved in the formation of a disulphide bridge.

25. The fusion protein according to claim 23, characterized in that it comprises allergens Parj1 and Parj2 of the Parietaria judaica species.

26. The fusion protein according to claim 25, characterized in that the amino acid sequence of each of the allergens is independently mutated by elimination or substitution of one or more cysteine residues in positions corresponding to the positions 4, 14, 29, 30, 50, 52, 75 and 91 of the amino acid sequence of Parj1 and/or Parj2 allergen.

27. The fusion protein according to claim 26, characterized in that it contains amino acid sequences of Parj1 and Parj2 allergens, both independently modified by substitution of cysteine residues with Asn, Ser, Thr, Ile, Met, Gly, Ala, Val, Gln or Leu residues in positions 29 and 30 or 4, 29 and 30 or 29, 30, 50, 52.

28. The fusion protein according to claim 27, comprising the amino acid sequence SEQ ID NO: 4.

29. A nucleotide sequence comprising the DNA coding for the fusion protein according to claim 25.

30. The nucleotide sequence according to claim 29 comprising the nucleotide sequence SEQ ID NO: 3.

31. An expression or cloning system comprising the nucleotide sequence according to claim 30 flanked by suitable sequences for controlling, promoting and regulating the expression.

32. A host cell transformed by means of the expression or cloning system according to claim 31.

33. The fusion protein according to claim 25, for use in a diagnostic or therapeutic treatment method in vivo and/or in vitro.

34. The fusion protein according to claim 33, for use as a hypoallergenic immunologic agent in the specific immunotherapy (SIT) treatment of allergies.

35. The fusion protein according to claim 33, for use in treatment of rhinitis, conjunctivitis, urticaria, angioedema, eczema, dermatitides, asthma, or anaphylactic shock.

36. The fusion protein according to claim 33, for preparation of a DNA vaccine.

37. A pharmaceutical composition comprising the fusion protein according to claim 25 and a pharmaceutically acceptable excipient.

38. The pharmaceutical composition according to claim 37 in the form of a solution, suspension, emulsion, cream, ointment or implant.

39. The pharmaceutical composition according to claim 37, for parenteral, subcutaneous, intramuscular, intravenous, topical, oral administration or for subcutaneous implantation.

40. A method of preparation of the fusion protein according to claim 25, characterized in that suitably mutated amino acid sequences of different allergens are produced and linked directly or via a spacer for chemical synthesis or by expression, in the form of fusion protein, in a genetically modified host cell.

41. The method of preparation according to claim 40, characterized in that a host cell is transformed with an expression vector comprising DNA coding for the amino acid sequences in fused form, which is mutated via site-specific mutagenesis in one or more codons coding for one or more cysteine residues.

42. The method of preparation according to claim 41, characterized in that one or more cysteine residues are substituted with Asn, Ser, Thr, Ile, Met, Gly, Ala, Val, Gln or Leu residues.

43. The method of preparation according to claim 40, characterized in that one or more cysteine residues in position 29 and 30; 4, 29 and 30; or 29, 30, 50 and 52 are substituted with alanine or serine residues.

44. The method of preparation of a pharmaceutical composition according to claim 37, characterized in that the heterodimer protein is mixed in an immunologically active amount to a pharmaceutically acceptable excipient.


SEQUENCE LISTING (7 sequences)

SEQ ID NO: 1 - DNA, 732 bp, Parietaria judaica
  CDS (1)..(729)
  misc_feature (10)..(12): n is a, c, g, or t
  misc_feature (40)..(42): n is a, c, g, or t
  misc_feature (85)..(90): n is a, c, g, or t
  misc_feature (148)..(150): n is a, c, g, or t
  misc_feature (154)..(156): n is a, c, g, or t
  misc_feature (223)..(225): n is a, c, g, or t
  misc_feature (271)..(273): n is a, c, g, or t
  misc_feature (322)..(324): n is a, c, g, or t
  misc_feature (352)..(354): n is a, c, g, or t
  misc_feature (397)..(402): n is a, c, g, or t
  misc_feature (460)..(462): n is a, c, g, or t
  misc_feature (466)..(468): n is a, c, g, or t
  misc_feature (535)..(537): n is a, c, g, or t
  misc_feature (583)..(585): n is a, c, g, or t
gaggaggctnnngggaaagtggtgcaggatataatgccgnnnctgcatttcgtgaagggggaggagaaggagccgtcgaaggagnnnnnnagcggcacgaagaagctgagcgaggaggtgaagacgacggagcagaagagggaggccnnnaagnnnatagtgcgcgccacgaagggcatctccggtatcaaaaatgaacttgtcgccgaggtccccaagaagnnngatattaagaccactctcccgcccatcaccgccgacttcgactgcnnnaagatccaaagtactattttcagaggttactatggattccaagaaaccnnngggactatggtgagagcgctgatgccgnnnctgccgttcgtgcaggggaaagagaaagagccgtcaaaggggnnnnnnagcggcgccaaaagattggacggggagacgaagacggggccgcagagggtgcacgctnnngagnnnatccagaccgccatgaagacttattccgacatcgacgggaaactcgtcagcgaggtccccaagcacnnnggcatcgttgacagcaagctcccgcccattgacgtcaacatggacnnnaagacagttggagtggttcctcggcaaccccaacttccagtctctctccgtcatggtcccgtcacgggcccaagtgatcccgcccacaaagcacggttggagagaccccagattagagttccgccccccgcaccggaaaaagcctaa

SEQ ID NO: 2 - protein, 243 aa, Parietaria judaica
  misc_feature (4)..(4): Xaa at location 4 stands for Lys, Asn, Arg, Ser, Thr, Ile, Met, Glu, Asp, Gly, Ala, Val, Gln, His, Pro, Leu, Tyr, Trp, Cys or Phe
  misc_feature (14)..(14): Xaa at location 14 stands for Lys, Asn, Arg, Ser, Thr, Ile, Met, Glu, Asp, Gly, Ala, Val, Gln, His, Pro, Leu, Tyr, Trp, Cys or Phe
  misc_feature (29)..(29): Xaa at location 29 stands for Lys, Asn, Arg, Ser, Thr, Ile, Met, Glu, Asp, Gly, Ala, Val, Gln, His, Pro, Leu, Tyr, Trp, Cys or Phe
  misc_feature (30)..(30): Xaa at location 30 stands for Lys, Asn, Arg, Ser, Thr, Ile, Met, Glu, Asp, Gly, Ala, Val, Gln, His, Pro, Leu, Tyr, Trp, Cys or Phe
  misc_feature (50)..(50): Xaa at location 50 stands for Lys, Asn, Arg, Ser, Thr, Ile, Met, Glu, Asp, Gly, Ala, Val, Gln, His, Pro, Leu, Tyr, Trp, Cys or Phe
  misc_feature (52)..(52): Xaa at location 52 stands for Lys, Asn, Arg, Ser, Thr, Ile, Met, Glu, Asp, Gly, Ala, Val, Gln, His, Pro, Leu, Tyr, Trp, Cys or Phe
  misc_feature (75)..(75): Xaa at location 75 stands for Lys, Asn, Arg, Ser, Thr, Ile, Met, Glu, Asp, Gly, Ala, Val, Gln, His, Pro, Leu, Tyr, Trp, Cys or Phe
  misc_feature (91)..(91): Xaa at location 91 stands for Lys, Asn, Arg, Ser, Thr, Ile, Met, Glu, Asp, Gly, Ala, Val, Gln, His, Pro, Leu, Tyr, Trp, Cys or Phe
  misc_feature (108)..(108): Xaa at location 108 stands for Lys, Asn, Arg, Ser, Thr, Ile, Met, Glu, Asp, Gly, Ala, Val, Gln, His, Pro, Leu, Tyr, Trp, Cys or Phe
  misc_feature (118)..(118): Xaa at location 118 stands for Lys, Asn, Arg, Ser, Thr, Ile, Met, Glu, Asp, Gly, Ala, Val, Gln, His, Pro, Leu, Tyr, Trp, Cys or Phe
  misc_feature (133)..(133): Xaa at location 133 stands for Lys, Asn, Arg, Ser, Thr, Ile, Met, Glu, Asp, Gly, Ala, Val, Gln, His, Pro, Leu, Tyr, Trp, Cys or Phe
  misc_feature (134)..(134): Xaa at location 134 stands for Lys, Asn, Arg, Ser, Thr, Ile, Met, Glu, Asp, Gly, Ala, Val, Gln, His, Pro, Leu, Tyr, Trp, Cys or Phe
  misc_feature (154)..(154): Xaa at location 154 stands for Lys, Asn, Arg, Ser, Thr, Ile, Met, Glu, Asp, Gly, Ala, Val, Gln, His, Pro, Leu, Tyr, Trp, Cys or Phe
  misc_feature (156)..(156): Xaa at location 156 stands for Lys, Asn, Arg, Ser, Thr, Ile, Met, Glu, Asp, Gly, Ala, Val, Gln, His, Pro, Leu, Tyr, Trp, Cys or Phe
  misc_feature (179)..(179): Xaa at location 179 stands for Lys, Asn, Arg, Ser, Thr, Ile, Met, Glu, Asp, Gly, Ala, Val, Gln, His, Pro, Leu, Tyr, Trp, Cys or Phe
  misc_feature (195)..(195): Xaa at location 195 stands for Lys, Asn, Arg, Ser, Thr, Ile, Met, Glu, Asp, Gly, Ala, Val, Gln, His, Pro, Leu, Tyr, Trp, Cys or Phe
EEAXGKVVQDIMPXLHFVKGEEKEPSKEXXSGTKKLSEEVKTTEQKREAXKXIVRATKGISGIKNELVAEVPKKXDIKTTLPPITADFDCXKIQSTIFRGYYGFQETXGTMVRALMPXLPFVQGKEKEPSKGXXSGAKRLDGETKTGPQRVHAXEXIQTAMKTYSDIDGKLVSEVPKHXGIVDSKLPPIDVNMDXKTVGVVPRQPQLPVSLRHGPVTGPSDPAHKARLERPQIRVPPPAPEKA

SEQ ID NO: 3 - DNA, 732 bp, Parietaria judaica
  CDS (1)..(729)
gaggaggctagcgggaaagtggtgcaggatataatgccgtgcctgcatttcgtgaagggggaggagaaggagccgtcgaaggagagcagcagcggcacgaagaagctgagcgaggaggtgaagacgacggagcagaagagggaggcctgcaagtgcatagtgcgcgccacgaagggcatctccggtatcaaaaatgaacttgtcgccgaggtccccaagaagtgcgatattaagaccactctcccgcccatcaccgccgacttcgactgctccaagatccaaagtactattttcagaggttactatggattccaagaaaccagcgggactatggtgagagcgctgatgccgtgcctgccgttcgtgcaggggaaagagaaagagccgtcaaaggggagcagcagcggcgccaaaagattggacggggagacgaagacggggccgcagagggtgcacgcttgtgagtgcatccagaccgccatgaagacttattccgacatcgacgggaaactcgtcagcgaggtccccaagcactgcggcatcgttgacagcaagctcccgcccattgacgtcaacatggactgcaagacagttggagtggttcctcggcaaccccaacttccagtctctctccgtcatggtcccgtcacgggcccaagtgatcccgcccacaaagcacggttggagagaccccagattagagttccgccccccgcaccggaaaaagcctaa

SEQ ID NO: 4 - protein, 243 aa, Parietaria judaica
EEASGKVVQDIMPCLHFVKGEEKEPSKESSSGTKKLSEEVKTTEQKREACKCIVRATKGISGIKNELVAEVPKKCDIKTTLPPITADFDCSKIQSTIFRGYYGFQETSGTMVRALMPCLPFVQGKEKEPSKGSSSGAKRLDGETKTGPQRVHACECIQTAMKTYSDIDGKLVSEVPKHCGIVDSKLPPIDVNMDCKTVGVVPRQPQLPVSLRHGPVTGPSDPAHKARLERPQIRVPPPAPEKA

SEQ ID NO: 5 - DNA, 18 bp, Artificial Sequence
  forward primer for insertion of mutation in position 29 and 30
gagagcagcagcggcagc

SEQ ID NO: 6 - DNA, 30 bp, Artificial Sequence
  forward primer for insertion of mutation in position 4
gtgggatccgaggaggctagcgggaaagtg

SEQ ID NO: 7 - DNA, 24 bp, Artificial Sequence
  reverse Parj2 primer
gggggatccatagtaacctctgaa